(19) United States
(12) Patent Application Publication
(10) Pub. No.: US 2022/0055073 A1
(43) Pub. Date: Feb. 24, 2022

(54) CLEANING PROCESS AND SYSTEM FOR PERFORMING THE SAME

(71) Applicant: Dynamo Industries, Inc., Plantagenet (ON) (CA)

(72) Inventors: Richard MARTIN, Cumberland (ON) (CA); Luc MARTIN, Gatineau (QC) (CA); Melissa GRANT, Ottawa (ON) (CA)

(21) Appl. No.: 17/408,719

(22) Filed: Aug. 23, 2021

Related U.S. Application Data
(60) Provisional application No. 63/069,640, filed on Aug. 24, 2020.

(51) Int. Cl. B08B 3/08; B08B 3/02; A61L 2/18

(52) U.S. Cl. CPC B08B 3/08; B08B 3/026; A61L 2202/17; B08B 2230/01; B08B 2203/007; A61L 2/18

(57) ABSTRACT

A process for cleaning an object, such as for instance an outdoor playground structure or other communal object, includes applying a degreaser solution to a surface of the object. The surface is also power washed using water at or above ambient temperature, and additionally steam is applied to the surface. A disinfectant solution is applied to the surface, and if a final contaminant level of the surface is below a predetermined threshold value then finally an antimicrobial product is applied. The process removes contaminants including microorganisms and organic residues and provides a surface that remains resistant to recontamination for an extended period of time.

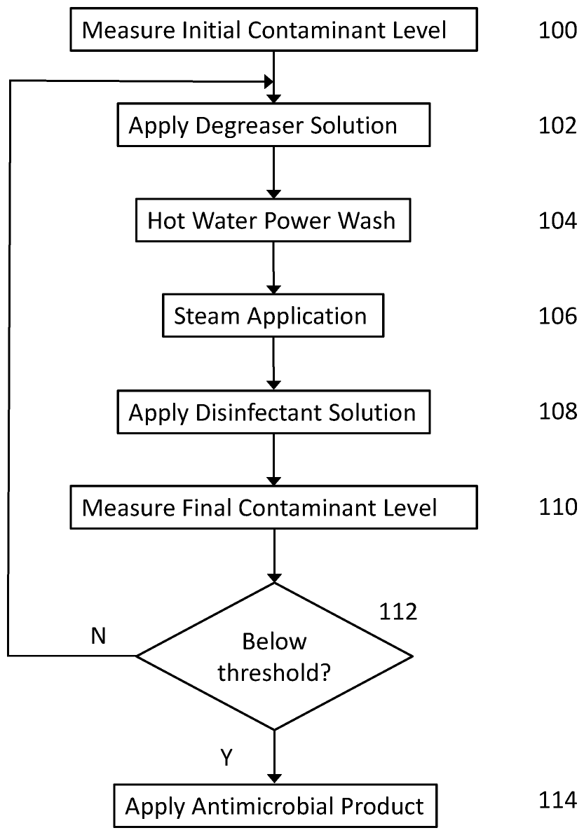

### FIELD OF THE INVENTION

The present disclosure relates generally to the field of cleaning or sterilization, and more particularly to processes and systems for cleaning and/or sterilizing objects such as playground structures and other communal objects.

### BACKGROUND

Even before the emergence of the SARS-CoV-2 novel coronavirus in December 2019, indoor and outdoor play structures were known to be significant transmission vectors for bacterial and viral infections. In fact, surface contamination on such play structures often includes micro-organisms, excrement, body fluid, blood and food residue, etc. Unfortunately, exposure to sunlight and rain does not significantly reduce the level of contamination or the risk of transmission by hand contact on outdoor structures, and therefore proper cleaning and/or sanitizing practices are very important, regardless of where the structures are located.

Many cleaning procedures merely remove or reduce the amount of dirt, dried fluids and other aesthetically displeasing substances from plastic and metal surfaces of play structures. Unfortunately, such procedures do not necessarily address the contamination that is caused by bacteria and/or viruses, which are not visible to the naked eye. Such procedures may include the application of soaps, even disinfecting soaps, by spraying and/or hand scrubbing. However, even using labor intensive procedures that require the structures to be partially disassembled it is still possible that certain inaccessible areas will not be cleaned and/or some accessible areas will not be cleaned sufficiently well. Part of the problem is that the initial and final levels of bacterial and viral contamination on the structure are not known, and therefore the cleaning activity may be stopped before a desired or required level of cleanliness is achieved.

Another problem that is associated with known cleaning procedures, especially in the context of high-traffic play structures, relates to re-contamination after the cleaning process has been completed. A play structure that is cleaned one day may have a high level of contamination the next day or even later the same day. It is therefore important to consider the frequency, and the cumulative cost, of cleaning activity that is required to maintain a safe play area.

Of course, since young children seldom practice adequate handwashing hygiene and are also prone to touching their face or putting their fingers in their mouths, it is undesirable to use harmful chemicals when cleaning play structures, even if the structures are thoroughly rinsed at the end of the cleaning process. On the other hand, mild soaps used for cleaning may not, on their own, eliminate sufficient amounts of bacteria and viruses on play equipment surfaces, which increases the risk of infections and necessitates more frequent cleanings.

It would therefore be beneficial to provide a process and a system that overcome at least some of the above-mentioned disadvantages and/or limitations.

### SUMMARY

In accordance with an aspect of at least one embodiment there is provided a process for cleaning an object, comprising:

a) applying a degreaser solution to a surface of the object;

b) power washing the surface using water at or above ambient temperature;

c) applying steam to the surface;

d) applying a disinfectant solution to the surface; and

e) using an electrostatic applicator, applying an antimicrobial product.

In accordance with an aspect of at least one embodiment there is provided a process for cleaning an object, comprising the following ordered steps:

a) obtaining a measure of an initial contaminant level of the surface;

b) applying a degreaser solution to a surface of the object;

c) power washing the surface using water at or above ambient temperature;

d) applying steam to the surface;

e) applying a disinfectant solution to the surface; and

f) obtaining a measure of a final contaminant level of the surface.

### BRIEF DESCRIPTION OF THE DRAWINGS

The invention will now be described by way of example only, and with reference to the attached drawings, wherein similar reference numerals denote similar elements throughout the several views, and in which:

The FIGURE is a simplified flow diagram showing a method according to an embodiment of the instant invention.

### DETAILED DESCRIPTION OF EMBODIMENTS OF THE INVENTION

The following description is presented to enable a person skilled in the art to make and use the disclosure and is provided in the context of a particular application and its requirements. The drawings are intended to be illustrative and are not drawn to scale. Various modifications to the disclosed embodiments will be readily apparent to those skilled in the art, and the general principles defined herein may be applied to other embodiments and applications without departing from the scope of the disclosure. Thus, the present disclosure is not intended to be limited to the embodiments disclosed but is to be accorded the widest scope consistent with the principles and features disclosed herein.

The use of water, “dry” steam, and various types of cleaning or disinfecting solutions is known for cleaning play structures and other communal objects. However, prior cleaning methods have been found to suffer a number of drawbacks that result in unsatisfactory results in terms of the extent to which surface contamination of an object is reduced as well as the length of time before before significant re-contamination of the object occurs.

A common cleaning procedure uses a simple bleach solution, which requires a minimum “touch time” on the surface to be effective to kill 99.99% of bacteria on the surface. The actual effectiveness of this procedure depends strongly on a variety of factors, and typically the observed effectiveness is far less than 99.99%. In order to demonstrate this fact, a simple test was performed by swabbing a surface of an outdoor playground structure both prior to and subsequent to spraying a simple bleach solution onto the surface. The samples were analyzed using a hygiene monitoring instrument (e.g., a LUMITESTER PD-30). Such an instrument provides an RLU (Relative Light Unit) value that is dependent on an amount of intra- and extracellular Adenosine Triphosphate (ATP) as well as Adenosine Monophosphate (AMP) in the swabbed sample. An ATP hygiene monitoring instrument provides a measurement of the direct risks resulting from high levels of microorganisms plus the indirect risks resulting from organic residues that can protect and provide a source of nutrients to microorganisms. As will be apparent, viruses are not detected using an ATP hygiene monitoring instrument because viruses do not contain ATP.

The test results that were obtained for the surface of the outdoor playground structure provided an initial measurement of ca. 600,000 RLU for the uncleaned surface and ca. 240,000 RLU for the same surface after the application of a bleach solution with a 10 minute “touch time.” Although it is not possible to make definitive conclusions about the risk of disease transmission after completing this simple cleaning procedure, it is clear that a substantial portion of the original contaminants still remain on the “cleaned” surface.

Additional testing was then performed to evaluate the effectiveness of other cleaning processes in combination with the application of the bleach solution. Using the same analysis techniques, it was found that pressure washing the test surface using cold water further decreased the measurement to ca. 70,000 RLU. Subsequent power washing using hot water (120° C.) and then using “dry hot steam” (120° C.) further reduced the measurement to ca. 45,000 RLU and ca. 12144 RLU, respectively. Finally, the spray application of a second sanitizer (EnviroNize® Daycare Disinfectant 500), which was subsequently allowed to dry for 2-3 minutes, produced a final measurement of ca. 270 RLU. For the purpose of this disclosure, a measurement of less than about 1000 RLU is considered to indicate a successful cleaning and sanitizing of a surface.

It has now been discovered that a combination of steps is required to clean outdoor play structures, or indoor play structures and other communal objects, in a way that achieves a sufficient removal of surface contaminants and that equally importantly results in a lasting level of cleanliness that reduces the need for frequent cleaning. For instance, a cleaning interval of at least two weeks, preferably at least one month, and more preferably longer than one month.

A general method according to an embodiment is illustrated in the FIGURE. The steps are shown in the FIGURE and are described below in a specific order that has been found to be particularly convenient and effective in cleaning and sanitizing surfaces such as for instance the surfaces of outdoor playground structures. However, strict adherence to the order of the steps disclosed with reference to the FIGURE is not essential. Some steps may be performed in a different order, and optionally some steps may be performed more than one time in a sequence.

At step 100 a measurement is made of the initial contaminant level. This measurement optionally includes separate measurements that are specific for different pathogens. However, it is sufficient to measure only a “total” initial contaminant level using e.g. the above mentioned LUMITESTER PD-30. Such a measurement is indicative of the presence of microorganisms (other than viruses) as well as organic residues that can protect and provide a source of nutrients to the microorganisms. Step 100 may include swabbing one or more representative sections of the surface to be cleaned using special sample swabs, reacting the swabbed material with a predetermined reagent to obtain an analyzable sample, and measuring luminescence of the analyzable sample to obtain a measurement in RLU. During real world testing, initial contaminants level measurements in the range ca. 600,000 RLU to greater than 999,999 RLU were obtained.

A degreaser solution is then applied to the surface at step 102. A specific and non-limiting example is EnviroNize® Catholyte, which is a high pH solution (11.5-13) used as a detergent or cleaning agent. The application of the degreaser solution is preferably done by electrostatic application, for instance using an EMist 360 series electrostatic sprayer. The electrostatic sprayer system places an electrical charge on the droplets of the degreaser solution and disperses them across a target surface area, providing a comprehensive, even coverage. This provides a consistent and uniform coverage in which the droplets adhere to vertical, horizontal and three-dimensional surfaces and “wraps” around the target area. Advantageously, the application time is minimized since it is not necessary to walk around the play structure and spray it from different sides, nor is it necessary to disassemble the play structure to cover hard-to-reach areas.

The surfaces are power washed at step 104 using hot, high pressure water. By way of a specific and non-limiting example, 120° C. water is sprayed at a pressure of 3500 psi. Advantageously, the water is hot enough and the pressure is high enough to kill microorganisms, possibly a majority of the microorganisms that are initially present, and assists with the removal of dirt, grease and other biological residues.

At step 106 the surfaces are then sprayed with hot “dry” steam at a temperature of about 120° C. to perform a “deeper kill” that is facilitated by the previous removal grease, dirt and other residues etc. from the surfaces.

At step 108 the surfaces are sprayed with a disinfectant solution. Preferably, the disinfectant solution achieves a kill rate of 99.99%. By way of a specific and non-limiting example, EnviroNize 500 Anolyte is used as the disinfectant solution, which is extremely effective and also ecologically safe and non-toxic for people, animals and for the environment. Advantageously, EnviroNize 500 Anolyte requires a touch time of only 1 minute for 99.99% kill rate, which ensures that the product does not fully dry on the possibly hot playground structure surfaces, which may also be exposed to wind, before it has finished killing to the rate of 99.99%.

A measurement is then made of the final contaminant level at step 110, subsequent to completing the cleaning/sanitizing steps 102 through 108. Preferably, the same representative surfaces are swabbed a second time using special sample swabs, and the swabbed material is reacted with a predetermined reagent to obtain an analyzable sample, and the luminescence of the analyzable sample is measured to obtain a measurement in RLU.

The final contaminant level measurement is then compared to the initial contaminant level at decision step 112 to determine, qualitatively, a pass or fail condition. Although various criteria may be set for determining whether or not a particular cleaning process has been successful, and therefore a pass condition, a convenient metric is based on achieving a final RLU value that is close to a target value for food hygiene testing. Since strict compliance with food hygiene standards is considered to be excessive, a suitable RLU range (as measured using the above mentioned LUMITESTER PD-30) is about 100 to about 1500, and more preferably about 500 to about 1000. Of course, criteria for establishing a pass condition may vary from customer to customer, and may vary depending on the type of surfaces being cleaned etc.

If it is determined at decision step 112 that a fail condition exists (i.e., a final contaminant level greater than about 1000 RLU is obtained), then additional cleaning steps are performed. As shown in the FIGURE, steps 102 through 110 are repeated. Optionally, only some of steps 102 through 110 are repeated. For instance, only steps 104 through 110 are repeated, or only steps 108 through 110 are repeated, etc. A second measurement of the final contaminant level is obtained when step 110 is repeated, which is then compared to the initial contaminant level at decision step 112. This process continues until a pass condition is achieved.

After completing the cleaning/sanitizing as described above, an antimicrobial product is applied onto the surfaces by spraying at step 114. A specific and non-limiting example of a suitable antimicrobial product is Zoono Z-71 Microbe Shield. This product is considered a mechanical kill and not a chemical kill. When applied to a surface by spraying, wiping or ‘fogging’ Zoono (or another similar product) leaves behind a mono-molecular layer that permanently bonds to the surface. These molecules are silane-based polymers that covalently bond to the surface forming a barrier of positively charged microscopic pins. The positively charged microscopic pins attract and pierce negatively charged microorganisms. The pins rupture the cell walls. This causes the microorganisms to break up with lethal effect. Although abrasion and other environmental factors will have an effect, it is reasonable to expect at least 30 days of protection against recontamination of the cleaned surfaces after an antimicrobial product (e.g., Zoono Z-71 Microbe Shield) has been applied, after which the cleaning/sanitizing process should be repeated.

Specific examples illustrating the effectiveness of the general method will now be discussed. In order to evaluate the effectiveness of each procedure tried, additional measurements of the surface contamination level were obtained following the performance of each step. Normally, it is not considered to be necessary to obtain the intermediate measurements of the surface contamination level.

### Example 1—Vital Oxide® (Chlorine Dioxide)

A vertically oriented surface area of an outdoor play structure was swabbed for several seconds. The surface consisted of a moderately rough-textured plastic material. Analysis of the test swab using a LUMITESTER PD-30 yielded an initial contaminant level value of about 630,000 RLU (step 100 of the FIGURE). A solution of Vital Oxide® (contains chlorine dioxide) was sprayed directly onto the tested surface and allowed to dry for 5 minutes (step 108 of the FIGURE). Vital Oxide® is an EPA-registered hospital disinfectant cleaner, mold killer, and odor eliminator that is effective at killing bacteria, viruses (including SARS-CoV-2), and mold yet is non-corrosive to treated articles. A swab test yielded a result of ca. 420,000 RLU. The surface was then power washed using cold (ambient) water (swab test ca. 30,000). A degreaser solution was then applied (step 102 of the FIGURE). The area was then power washed again using hot water at a temperature of ca. 120° C. (step 104 of the FIGURE), and then hot “dry” steam was applied at a temperature of ca. 120° C. A swab test yielded a result of ca. 550. Finally, an acid disinfectant was applied (step 108 of the FIGURE). A final swab test yielded a result of less than 300 (step 110 of the FIGURE). The final test swab result indicated a pass condition (decision step 112 of the FIGURE). In this test sequence, the application of an antimicrobial product was omitted.

### Example 2—Zoono

A vertically oriented surface area of an outdoor play structure was swabbed for several seconds. The surface consisted of a moderately rough-textured plastic material. Analysis of the test swab using a LUMITESTER PD-30 yielded an initial contaminant level value of about 995,000 RLU (step 100 of the FIGURE). A solution of a “mechanical kill” disinfectant and antimicrobial product, in this specific example Zoono Z-71 Microbe Shield, was sprayed directly onto the tested surface and allowed to dry for 5 minutes (step 108 of the FIGURE). When applied to a surface by spraying, wiping or ‘fogging,’ the Zoono mechanical kill product leaves behind a mono-molecular layer that permanently bonds to the surface. These molecules are silane-based polymers that covalently bond to the surface forming a barrier of positively charged microscopic pins. The positively charged microscopic pins attract and pierce negatively charged microorganisms. The pins rupture the cell walls. This causes the microorganisms to break up with lethal effect. A swab test yielded a result of ca. 400,000 RLU. The surface was then power washed using cold (ambient) water (swab test ca. 5,000 RLU). The area was then power washed again using hot water at a temperature of ca. 120° C. (step 104 of the FIGURE), and then hot “dry” steam was applied at a temperature of ca. 120° C. (step 106 of the FIGURE). A swab test yielded a result of ca. 15,000 RLU. It is believed that the higher swab test result was caused by contamination from an adjacent area; only a small portion of the play structure was cleaned, and it is likely that contaminated water from the power washing steps simply flowed downward and into the test area. Finally, a second application of the Zoono product was applied and allowed to dry (step 114 of the FIGURE). A final swab test yielded a result of about 900 (step 110 of the FIGURE). The final test swab result indicated a pass condition (decision step 112 of the FIGURE).

As will be apparent, excellent results are achieved using the method that is described with reference to the FIGURE, even if some steps are performed in a different order.

In the description of the invention herein, it is understood that a word appearing in the singular encompasses its plural counterpart, and a word appearing in the plural encompasses its singular counterpart, unless implicitly or explicitly understood or stated otherwise. For instance, unless the context indicates otherwise, a singular reference, such as “a” or “an” means “one or more”. Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise. It is also to be understood, where appropriate, like reference numerals may refer to corresponding parts throughout the several views of the drawings for simplicity of understanding.

Throughout the description and claims of this specification, the words “comprise”, “including”, “having” and “contain” and variations of the words, for example “comprising” and “comprises” etc., mean “including but not limited to”, and are not intended to (and do not) exclude other components.

It will be appreciated that variations to the foregoing embodiments of the invention can be made while still falling within the scope of the invention. Each feature disclosed in this specification, unless stated otherwise, may be replaced by alternative features serving the same, equivalent or similar purpose. Thus, unless stated otherwise, each feature disclosed is one example only of a generic series of equivalent or similar features.

The use of any and all examples, or exemplary language (“for instance”, “such as”, “for example”, “e.g.” and like language) provided herein, is intended merely to better illustrate the invention and does not indicate a limitation on the scope of the invention unless otherwise claimed. No language in the specification should be construed as indicating any non-claimed element as essential to the practice of the invention.

Any steps described in this specification may be performed in any order or simultaneously unless stated or the context requires otherwise.

All of the features disclosed in this specification may be combined in any combination, except combinations where at least some of such features and/or steps are mutually exclusive. In particular, the preferred features of the invention are applicable to all aspects of the invention and may be used in any combination. Likewise, features described in non-essential combinations may be used separately (not in combination).

What is claimed is:

1. A process for cleaning an object, comprising:
a) applying a degreaser solution to a surface of the object;
b) power washing the surface using water at or above ambient temperature;
c) applying steam to the surface;
d) applying a disinfectant solution to the surface; and
e) using an electrostatic applicator, applying an antimicrobial product.

2. The process of claim 1, further comprising obtaining a measure of an initial contaminant level of the surface prior to step a).

3. The process of claim 2, further comprising obtaining a measure of a final contaminant level of the surface after step d).

4. The process of claim 3, comprising comparing the measure of the final contaminant level of the surface to a predetermined threshold value, and repeating at least some of steps a) to d) if the measure of the final contaminant level of the surface exceeds the predetermined threshold value.

5. The process of claim 1, wherein step d) includes allowing a touch time of at least one minute to elapse after applying the disinfectant solution to the surface before obtaining a measure of a final contaminant level of the surface.

6. The process of claim 1, wherein step b) is performed using water a temperature of at least 120° C. and at a pressure of at least 3500 psi.

7. The process of claim 1, wherein step c) is performed using steam having a temperature of at least 120° C.

8. The process of claim 1, wherein the antimicrobial product is a mechanical kill product.

9. A process for cleaning an object, comprising the following ordered steps:
a) obtaining a measure of an initial contaminant level of the surface;
b) applying a degreaser solution to a surface of the object;
c) power washing the surface using water at or above ambient temperature;
d) applying steam to the surface;
e) applying a disinfectant solution to the surface; and
f) obtaining a measure of a final contaminant level of the surface.

10. The process of claim 9, comprising comparing the measure of the final contaminant level of the surface to a predetermined threshold value, and repeating at least some of steps b) to e) if the measure of the final contaminant level of the surface exceeds the predetermined threshold value.

11. The process of claim 9, comprising comparing the measure of the final contaminant level of the surface to a predetermined threshold value, and if the measure of the final contaminant level of the surface is less than the predetermined threshold value applying an antimicrobial product to the surface using an electrostatic applicator.

12. The process of claim 11, wherein the antimicrobial product is a mechanical kill product.

13. The process of claim 9, wherein step e) includes allowing a touch time of at least one minute to elapse after applying the disinfectant solution to the surface before performing step f).

14. The process of claim 9, wherein step c) is performed using water a temperature of at least 120° C. and at a pressure of at least 3500 psi.

15. The process of claim 9, wherein step d) is performed using steam having a temperature of at least 120° C.
